(19) United States
(12) Patent Application Publication
(10) Pub. No.: US 2017/0169716 A1
(43) Pub. Date: Jun. 15, 2017

(54) SYSTEM AND METHOD FOR ASSESSING VISUAL AND NEURO-COGNITIVE PROCESSING

(71) Applicants: Selwyn Super, Los Angeles, CA (US); Adam Cegielski, Oakville (CA)

(72) Inventors: Selwyn Super, Los Angeles, CA (US); Adam Cegielski, Oakville (CA)

(21) Appl. No.: 15/389,757

(22) Filed: Dec. 23, 2016

Related U.S. Application Data
(63) Continuation of application No. 14/108,871, filed on Dec. 17, 2013, now abandoned.

(51) Int. Cl. G09B 7/00; A61B 3/08; A61B 3/028; A61B 5/16; A61B 5/00

(52) U.S. Cl. CPC G09B 7/00; A61B 5/162; A61B 5/4088; A61B 3/028; A61B 3/08

(57) ABSTRACT

A system and method for assessing visual and neuro-cognitive processing is provided that involves presenting questions on a video display of a computing device and receiving input from a test subject identifying the location of an item in each question. The response can be received by activating a button, voice input or a combination of the two. The time period from presenting the question to receiving the response is measured along with the accuracy of the response. The test can further involve stereo acuity by using stereoscopic glasses and having the test subject identify the object have stereo disparity such that it stands out (or back) from a reference plane. The testing integrates visual input, mental processing, and speech motor and/or hand-eye (or eye-foot) coordination inputs and measures the reaction time taken to complete these circuitries.

### FIELD

The present disclosure relates generally to systems and methods for combining and integrating sensorimotor, perceptual and cognitive testing of human subjects.

### BACKGROUND

Mental chronometry is a measure of cognitive speed and is the actual time taken to process information of different types and degrees of complexity. The basic measurement is the individual's response time (RT) to a visual or auditory stimulus that calls for a particular response, choice or decision.

It is known that many and various screening tests are available to test a subject's vision, hearing, speech, motor, and/or hand and motor coordination, as well as perceptual and cognitive skills. Some of these tests involve complex ideas or include some type of cultural or educational bias making it difficult to administer the same universal test across ages, academic grade level, cultures and groups with minimal degrees of mental processing ability.

Although RT tests appear to be very simple compared to the typical items in psychological tests, they can prove to be of significant value in exposing individual differences related to their sensorimotor, perceptual and cognitive components. Galley & Galley (1999) describe the use of certain features of eye movements as a chronometric method for the study of intelligence quotient (IQ). However, although the method is remarkably simple and efficient, it calls for specialized instrumentation and computer programs.

The King-Devick (KD) test of oculomotility is a tool for evaluation of saccade, or fast movements of the eye, that consists of a series of test cards of numbers or letters. The test cards become progressively more difficult to read due to variability of spacing between the characters. Both errors in reading and speed of reading are included in deriving a score. Apart from being able to recognize and name numbers or letters in a left to right sequence, the test lacks for other, simple and considered important, perceptual and cognitive reading demands.

### SUMMARY

According to a first aspect, a method is provided for assessing visual and neuro-cognitive processing comprising presenting at least one question on a video display, the question requesting identification of an item position; receiving at least one response corresponding to the at least one question; measuring a time period from presenting the at least one question to receiving corresponding response; and measuring an accuracy of the response to the question based on correct identification of the item position.

In some aspects the method can further comprise providing feedback when the response is measured as incorrect. Other aspects can include presenting multiple questions and receiving multiple responses. A total time period from presenting questions to receiving responses can be measured. The item position can also be randomly generated for each of the questions. The response can be received by activating a button to measure eye/hand motor control, received by verbal response using a microphone, or a combination. The verbal response can also require a question number as well as the item position.

The item position can be any one of left, middle and right, and the response is any one of left, middle and right. The item itself can be any one of the words “left”, “right” and “middle” in the directional word sequence reading test or the item can be an object or geometric shape, such as a circle, in the circle location speed test. The item can also be an object having stereo disparity which positions must be identified using stereoscopic glasses synchronized with the video display as an object lying in front or behind the plane of regard.

In some aspects, the time period measured can then be compared to a normative value. The normative value can be based on age or other characteristics of the test subject that can be determined by a questionnaire. The questionnaire can also elicit answers related to learning disabilities or neurological dysfunction or traum, e.g. concussion history. The questionnaire can further elicit responses related to education level (e.g. level of schooling), learning ability, occupation, health and fitness status, disease and illness and injury status, scholastic achievement, intelligence quotient, and sporting ability.

According to a second aspect, a system is provided for assessing visual and neuro-cognitive processing, the system comprising a computing device having a video display, a processor and a memory, the memory storing instructions to configure the processor to: present at least one question on a video display, the question requesting identification of an item position; receive at least one response corresponding to the at least one question; measure a time period from presenting the at least one question to receiving corresponding response; and measure an accuracy of the response to the question based on correct identification of the item position.

In some aspects, the system can further comprise a network accessible score database, and the computing device further comprises a network interface to send a test score to the score database. The network accessible score database can have a public “normative” database and an “abnormal” database, and can also have a private database to store a test subject's historic test score. The network accessible score database can also have an interface to allow access to a subject's test scores.

### BRIEF DESCRIPTION OF THE DRAWINGS

For a better understanding of the various embodiments described herein and to show more clearly how they may be carried into effect, reference will now be made, by way of example only, to the accompanying drawings which show at least one exemplary embodiment, and in which:

FIG. 1 is a block diagram of a computing device;

FIG. 2A is an illustration of an embodiment of a directional word speed reading test;

FIG. 2B is an illustration of an embodiment of a circle speed reading test;

FIG. 2C is an illustration of an embodiment of a horizontal arrangement of test questions that can be used with any of the tests illustrated in FIGS. 2A-B;

FIG. 2D is an illustration of an embodiment of a stereo acuity test having one of the circles having a stereo disparity;

FIG. 3 is a flowchart illustrating a method of screening a test subject using a computing device; and

FIG. 4 is a block diagram of a system for providing access to a database of test results from the screening test.

### DESCRIPTION OF VARIOUS EMBODIMENTS

It will be appreciated that for simplicity and clarity of illustration, where considered appropriate, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing the implementations of various embodiments described herein.

The embodiments of the systems, devices and methods described herein may be implemented in hardware or software, or a combination of both. Some of the embodiments described herein may be implemented in computer programs executing on programmable computers, each computer comprising at least one processor, a computer memory (including volatile and non-volatile memory), at least one input device, and at least one output device. For example, and without limitation, the programmable computers may be a server class computer having multiple processors and at least one network interface card. Program code may operate on input data to perform the functions described herein and generate output data.

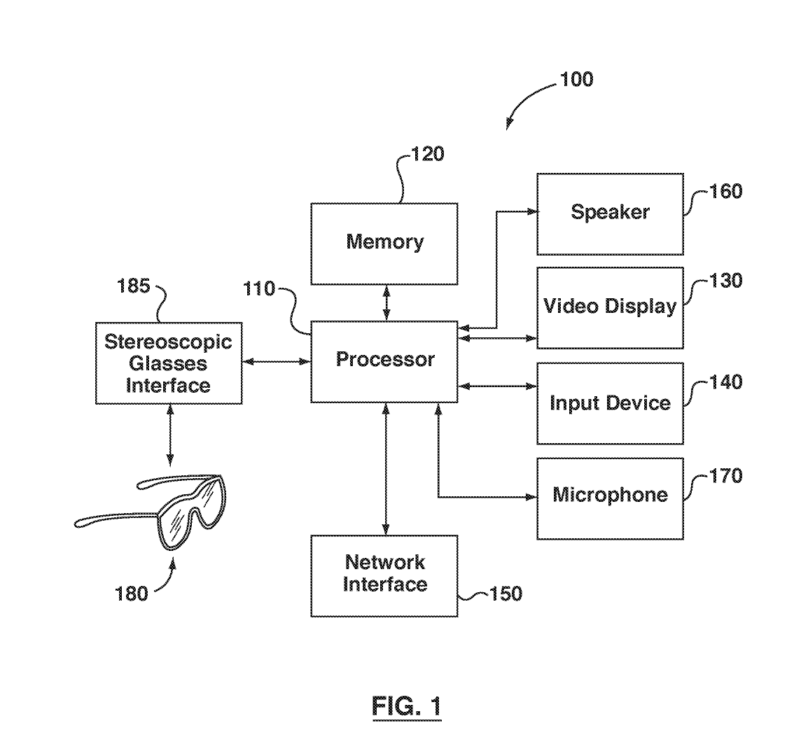

Reference is first made to FIG. 1, shown is a block diagram of a computing device 100 that can include a processor 110, memory 120, video display 130, and input device 140. A network interface 150 can be provided to allow computing device to communicate with other computing devices over a communication network. Computing device 100 can further include a speaker 160 and a microphone 170 for providing audio output and input. Examples of computing device 100 can include mobile computing devices, such as mobile phones, laptops or tablets, and computing devices used in the home, such as a desktop computer, video game console, set top box, or television.

Processor 110 is configured to control the operation of computing device 100, including coordination between other components coupled to processor 110. Control is provided by execution of software code stored in memory 120. Software code typically includes an operating system, such as Windows, Mac OS X, Linux, iOS, or Android, for example. Computing device 100 can also include any number of application programs stored in memory 120 and configured for execution by processor 110.

Processor 110 can include one or more programmable microprocessors or a microprocessor having more than one microprocessor core. In addition, processor 110 can include a central processing unit (CPU), memory (in addition to or such as the illustrated memory 120, such as a cache, for example) and an input/output interface through which processor 110 can receive a plurality of input/output signals. Some components of computing device 100 can be integrated with processor 110 and memory 120 in a system on a chip design.

Memory 120 can provide storage for data and software instructions for processor 110. Memory 120 can include both volatile and non-volatile memory. Non-volatile memory (i.e. non-transitory memory) can include flash memory or read-only memory including various forms of programmable read-only memory (e.g. PROM, EPROM, EEPROM). Volatile memory can include random access memory (RAM) including static random access memory (SRAM), dynamic random access memory (DRAM) and synchronous dynamic random access memory (SDRAM).

Video display 130 functions to provide a video output to user of computing device 100. Video display 130 can be a liquid crystal display, light emitting diode display, or other display technology known in the art. Video display 130 can also include an integrated input device 140, such as a digitizer, to provide a touchscreen interface to computing device 100. Input device 140 could also include other traditional input mechanisms, such as a keyboard and mouse, which can be used with computing device 100. Input device 140 could also be provided as a custom input device that provides larger buttons that correspond with the test responses. For example, a touch pad device or panel with buttons could be provided that included buttons for “left”, “middle” (or “center”), and “right”.

Video display 130 can also be used with stereoscopic glasses 180 in order to display stereograms to further test a subject's stereo acuity. Stereoscopic glasses 180 are preferably active shutter glasses that operate by alternatively blocking each eye and coordinating the blocking with video display 130 to provide alternating left and right eye images. Synchronization of video display 130 with active-shutter stereoscopic glasses 180 can be provided by stereoscopic glasses interface 185 that provides either a wired or wireless signal (e.g. infrared or radiofrequency, such as Bluetooth). The signal causes stereoscopic glasses 180 to synchronize with alternating left and right eye images provided by video display 130. Other embodiments can use passive stereoscopic systems that include different filters for each eye, such as polarization systems or color anaglyph systems, for example.

Stereoscopic glasses 180 can alternatively be used to block a single eye to perform monocular testing wherein the subject will be tested using only a single eye with the test images and then retested using the other eye. Synchronization with the glasses can be used to provide stereo crossed and uncrossed images.

In some embodiments, stereoscopic glasses 180 can include at least two markers that are separated by a known distance. Computing device 100 can further include a camera that can capture one or more images of the test subject wearing the stereoscopic glasses 180. Based on the distance separating the markers in the captured image and the known distance, processor 110 can calculate the distance between stereoscopic glasses 180 and video display 130. This measurement can be used to calculate degrees of arc based on the calculated distance and items on video display 130. Other embodiments can incorporate distances sensors (e.g. those based on infrared, sonar, or network time of flight) to calculate the distance between video display 130 and stereoscopic glasses 180. Some embodiments of computing device 100 can also include a gyroscopic sensor to help ensure that video display 130 is in a frontoparallel plane with stereoscopic glasses 180.

Speaker 160 is an electric to acoustic transducer that generates sound in response electrical signals provided by processor 110. Speaker 160 can be used to provide guidance and feedback to users of computing device 100. Microphone 170 converts audio signals into electrical signals that can be processed by processor 110. Microphone 170 can provide an alternative input mechanism that allows subjects of computing device 100 to use voice to provide instructions to computing device 100. In some embodiments, microphone 170 can be integrated with or attached to stereoscopic glasses 180.

Network interface 150 allows computing device 100 to connect to communication networks in order to communicate with other computing devices 100. These other computing devices 100 can be servers that are hosted on a local area network or are accessible over a publicly accessible network, such as the internet. Network interface 150 can be wired or wireless. Wireless network interfaces can include those that are compliant with Wi-Fi or Bluetooth standards, or a conventional cellular network interface.

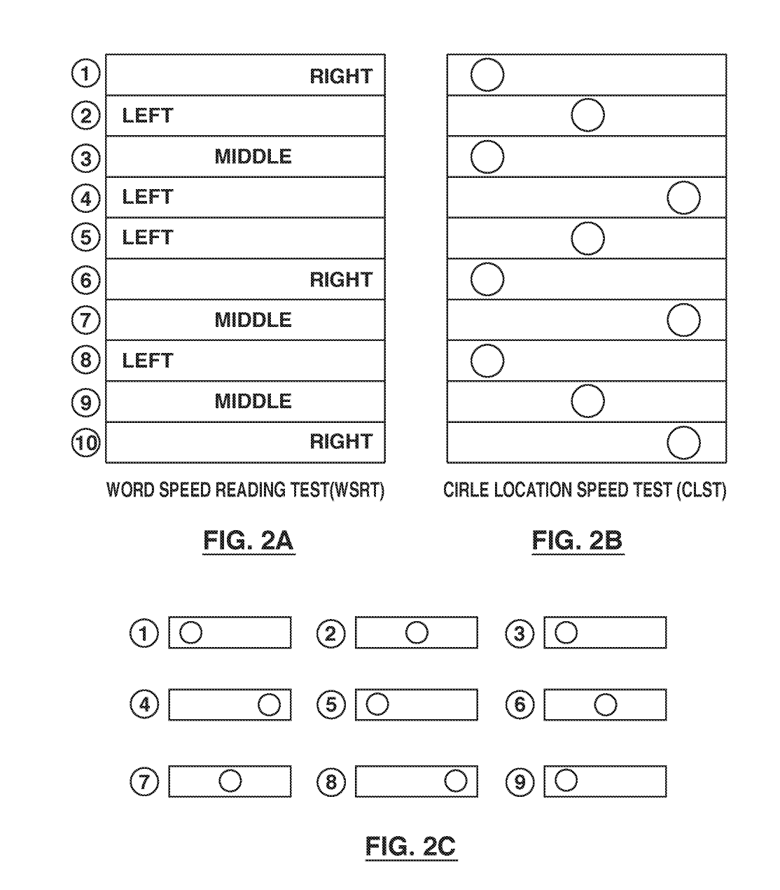

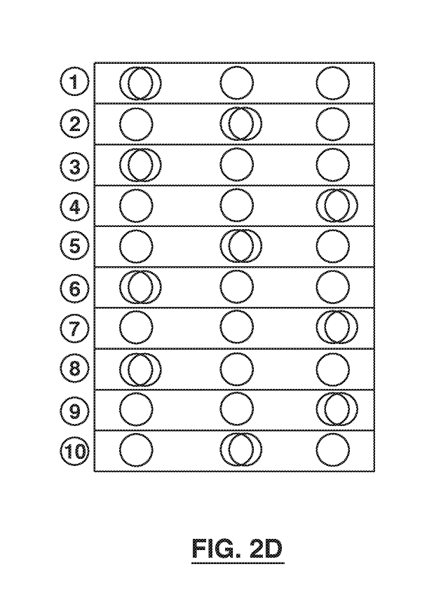

Referring now to FIGS. 2A-C, shown are illustrations of exemplary presentations of questions on video display 130 that request user of computing device 100 to identify an item position. The embodiment of FIG. 2A uses the words “left”, “middle”, and “right” as the item that can be located in a position of the rectangle. The embodiment of FIG. 2B uses the position of an object, such as the illustrated circle, within the rectangle for each of the questions. The embodiment of FIG. 2A is referred to herein as the Directional Word Speed Reading (DWSR) test and the embodiment of FIG. 2B is referred to herein as the Circle Location Speed (CLS) test. The embodiment of FIG. 2C illustrates an alternative horizontal arrangement of the questions that can be used with either the DSWR or CLS tests.

Computing device 100 can present questions, such as those illustrated in FIGS. 2A-C, on video display 130 to a user and receive a response from the user, such as through input device 140 or microphone 170, that identifies the position of the item. The speed to complete the test can be measured by computing device 100 as the time between presentation of the test on the video display and receiving the correct responses identifying the item positions.

The DWSR and CLS tests are two testing methodologies that can be used to assess the ability to read and/or recognize the spatial concepts of left, middle, and right. The speed with which this can be done has been discovered to be a developmental and age-related skill that is closely correlated to a person's intelligence quotient (IQ). The tests can be used to assess visual perception, neuro-cognitive function, gross and fine motor skills, and also vocal/speech function. The tests can be used to provide diagnostic information with regard to the function and coordination of the visual input and speech language systems. The DWSR and CLS tests the reaction time of the brain circuitry that combines essential sensorimotor, perceptual and cognitive systems of the human-subject at its most basic levels from the youngest possible age onwards.

The embodiments illustrated in FIGS. 2A-C use ten questions on the display at a time. The number of questions that are provided on the video display 130 can be varied in other embodiments to allow providing for a single question or multiple questions at a time. A test subject can be presented with more than ten questions on the display or provided multiple questions by refreshing the display. This can be used to test a subject's endurance to measure for any decline in the time to respond to the questions over time.

The item positions of the questions can be randomly generated to prevent a user from being able to memorize the item positions and the order of questions. It is also preferable that the randomization provides sufficient variation of the item position, such as to prevent an item appearing in the same position too often or too many times consecutively. Other embodiments can also repeat the same randomized questions in order to test memory.

In other embodiments of the DWSR test, the item position may not correspond with the word, thus requiring higher cognitive function from the test subject to correctly identify the item position. For example, the word “left” can appear in the middle position requiring the test subject to separate the concepts of item position with the item word.

The CLS test can be varied by using a different object other than the illustrated circle, such as other geometric shapes, pictograms. In some embodiments, the CLS test can include other objects as noise that can make it more difficult to ascertain the position of the desired item. For example, each question could include a star, a circle and a square, and the test subject would be requested to identify the position of the star.

Computing device 100 can be used in conjunction with stereoscopic glasses 180 to test a subject's stereo acuity. This can be used similar to the CLS test to determine the location of a circle that is displayed with stereo disparity. FIG. 2D illustrates ten questions in the form of stereograms that each present one circle in stereo disparity form and two circles in regular non-disparate form. FIG. 2D provides the combined left and right eye images overlaid together for ease of illustration. The position of the circle with stereo disparity differs in each question to minimize the risk of guessing a correct answer. The stereograms shown in FIG. 2D can vary the level of disparity (either per question or per test) to determine the speed of stereo acuity using changes in the level of disparity from 400 seconds and in non-parametric steps to the lowest number of seconds at a working distance of 40 cm and limited by test design.

Computing device 100 can direct the test subject to wear stereoscopic glasses for testing stereo acuity using the test illustrated in FIG. 2D. The test subject is instructed to select the circle, using voice or touch input for example, that stands out either in front or behind the reference plane in each question. The test may then be repeated with the stereoscopic glasses in the opposite mode. The circles with stereo disparity will be perceived to recede relative to the plane of fixation when using glasses in the uncrossed mode and to stand out in front of the reference ground when in the crossed mode.

Similar to the CLS test, the stereoscopic test can be varied to randomize the position of the circle with stereo disparity and with three different target designs, that is, single line contour (local stereopsis), random dot (global stereopsis), and with single line contour superimposed on random dot (local and global combined stereopsis) and with crossed disparity so as to see the stereoscopic target in front of the plane of regard, and with uncrossed disparity so as to see the stereoscopic target behind the plane of regard.. The background for the test can also vary to include varying amounts of noise, from a clear background to those including increased density of randomly spaced dots. The level of stereo disparity can also vary by question making it more difficult or easier to detect the stereo disparate circle in each question.

Measuring stereo acuity provides a more discriminative measure of visual acuity than can be provided using a Snellen chart. A measure of 20/10 vision based on a Snellen chart means that the subject can discern down to 150 seconds of arc. By testing stereo acuity using the test illustrated in FIG. 2D and stereoscopic glasses 180 can test more discrimatively and down to 3 seconds of arc depending on the resolution of video display 130 and the distance from stereoscopic glasses 180.

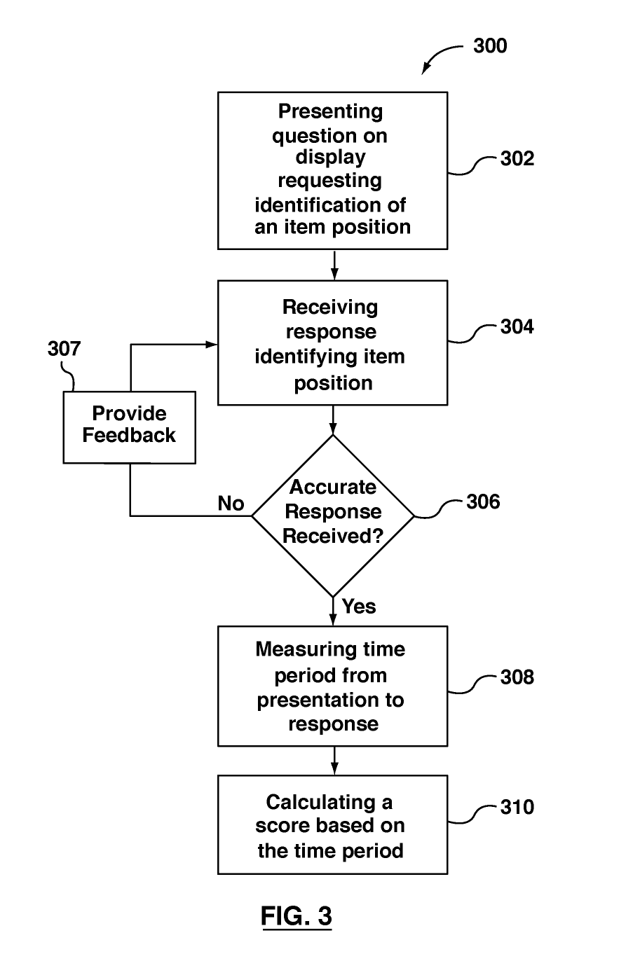

Referring now to FIG. 3, shown is a flow chart illustrating a method 300 for assessing visual and neuro-cognitive processing. Method 300 can be implemented as a software application executing on processor 110 of computing device 100. For example, method 300 can be provided by an application executing on a tablet or desktop computing device. The application can allow a user to select a language, provide instructions on how to complete the testing providing by the application, and provide a registration function, among other features. In some embodiments, the test subject can also be presented with a questionnaire that can be used to determine levels of symptomatology present. After the test subject has registered, been provided with and understood the instructions, the test subject can initiate the test, for example, by providing a “begin test” input, such through an onscreen button. Initiating the test can begin a countdown after which one or more questions will be presented to the test subject to provide identification of the item position in step 302.

At step 302, any of the DSWR or CLS tests, or variants thereof, described with respect to FIGS. 2A-D can be displayed on the video display 130. Method 300 provides a timed test and step 302 can further include starting a timer or recording a start time for the test upon presenting the first question on video display 130.

Preferably, method 300 is implemented using the DSWR test first followed by the CLS test. As provided in the instructions, display of these tests requires the test subject to correctly identify the item position for each of the questions as quickly as possible. Some embodiments can further include testing using stereoscopic glasses to include stereo acuity aspects (e.g. using a test similar to FIG. 2D) or monocular vision. Stereo acuity tests can use varying levels of stereo disparity (in terms of seconds of arc) to test stereo acuity.

Upon being presented with one or more questions on the video display 130, the test subject must provide answers as quickly as possible. Responses are received identifying the item position in each question in step 304. In the DWSR and CLS tests the test subject determines the position of the item (i.e. the word or object, respectively) and can provide input to the computing device.

The response can be received from input device 140, such as buttons on a touch screen or an external keyboard. A touch screen embodiment can include buttons labelled to represent “left”, “middle”, or “right”, for example. Tests carried out with buttons in this manner can provide an indication of visual and motor coordination. The term motor coordination is used herein to refer to hand-eye coordination, or the visual coordination of other body parts, such as the feet for example. In other embodiments, microphone 170 can be used to allow the test subject to vocalize the responses. For example, the test subject must state the question number and identify the item location (e.g. “one left, two middle, three right”). Some embodiments can use both verbal and hand coordinated input from the test subject. These different aspects can be tested sequentially or together so that the test subject must press a button and verbalize the response simultaneously.

Next, at step 306, the accuracy of the response received from the test subject is evaluated against the questions that were provided in step 302. Processor 110 compares the input received (e.g. from input device 140 and/or microphone 170) with the question to determine if the test subject provided the correct response. The accuracy determination of the received responses can be used in determining the test subject's score, or to request the test subject re-attempt the question which will extend the time it takes for the test subject to complete the test.

In some embodiments, if an incorrect response is received immediate feedback can be provided at step 307 that indicates that the test subject had provided an incorrect response and should make another attempt to provide the correct response. Feedback can be provided by video display 130, speaker 160, vibration motor, or any combination thereof. Examples could include a displaying a red “X” on the video display or making a buzzing sound from speaker 160. The subsequent response from the test subject would then be received at step 304 and re-evaluated at step 306.

At step 308, the time period is measured from the presentation of the one or more questions in step 302 to receiving a response in step 304. This can be used to measure the test subjects reaction time. The time period can be measured on a question by question basis, and the time period can also be measured for completion of multiple questions, such as all those displayed on video display 130 at one time or for the entire test. Measuring the time period for each question can measure the variance between answers to determine if the test subject is slowing down during progression of the test.

Next, at step 310, a score can be calculated for the test subject based on the measured time period(s) from step 308. Some embodiments can also use the accuracy determination from step 306 in calculating a score. Examples of calculating the score can include the overall time period for the test or average reaction time for question. Other factors can be used to weight the score, such as whether the test subject's reaction times were increasing or decreasing throughout the test. The calculated score can separate hand/eye input and verbal input, or provide a combined score using both.

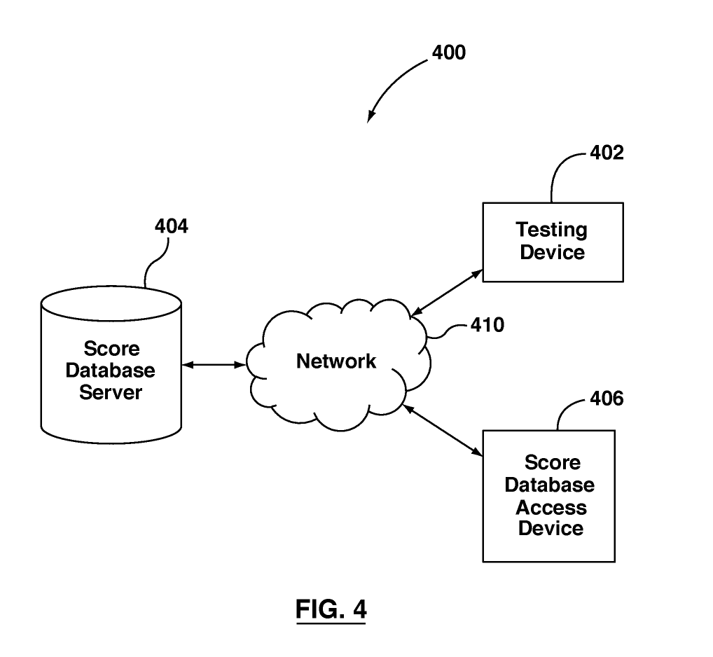

The score calculated in step 310 can be compared against the test subject's historic scores or normative values determined from test results of the general population. Computing device 100 can include a table of scores that can be used for comparison purposes with the test subject's score. In other embodiments, a score database hosted on a network, such as that described with respect to FIG. 4, can be used to evaluate the test results. The table of scores can be indexed by age to determine if the test subject's results are normal for their group. The grouping can include age, scholastic grade level, age and sex, or age, sex and geographic location, among other factors and combinations.

If it is determined that the test results are outside of the normative values a questionnaire can be provided to the test subjects to determine possible causes for the variance. The questionnaire can provide questions that relate to presence of learning disabilities, the possible occurrence of neurological trauma (e.g. a concussion), or other symptomatology.

Research has shown the DWSR and CLS results to be significantly related to individuals' ages, grades at school and to non-verbal and verbal IQs with very high probability (e.g. P values of 0.0001). The claim is made, therefore, assuming there are no errors of measurement, that the DWSR and CLS are measures of neurological integrity that demonstrate and correlate with higher IQs when the test subjects' performance speeds are faster than those expected for their ages or grades at school. However, when subjects' performance speeds on the DWSR and CLS are slower than those expected for their ages or grades at school, the causes can be numerous. The slowness may result from one or more of a combination of lack of understanding of the tasks required, poor motivation, poor attention span, poor directionality, poor visual acuity, poor ocular motility, poor binocular vision, poor eye-hand coordination, poor language and speech articulation, developmental delay or low IQ.

The value of the DWSR and CLS tests lie in their simplicity of design and very short time of testing (seconds rather than minutes) and their repeatability, which show training and memory effects. The tests show the importance of knowing the right direction and moving there, which in turn involves spatiotemporal orientation and actions directed by the ability to pinpoint objectives (top down brain functions) and to pinpoint objects in spacetime (bottom up body and brain functions). These are crucial to the ways human beings act, function and behave.

One valuable aspect of the present invention results in using computing device 100 to measure millions of subjects and to accumulate and process a large database that can be standardized and against which individual differences and performances may be assessed regarding health and disease states, academic and sporting performances, and training and learning protocols. In qualified hands, the test therefore should prove to be of significant diagnostic value.

Referring now to FIG. 4, shown is a block diagram of a system 400 for providing access to a test score database server 404 from any one of testing device 402 and score database access device 406 over communication network 410. Testing device 402 can be a computing device 100 that implements that the screening method described in FIG. 3. Score database server 404 and access device 404 can also be implemented as computing devices 100, each including a network interface 150 to allow each of the devices in FIG. 4 to communicate over network 410.

Testing device 402 can allow a test subject to register and setup an account with the score database server 404. Score database 404 can store account records for each test subject that track aspects such as age, sex, geographic location, and other aspects that may be relevant to a test subject's test score. Score database server 404 can provide authenticated access using the test subject's user credentials to obtain secure access (e.g. encrypted) between testing device 402 or access device 406. Score database server 404 can also allow a test subject to share their test results with the account of a health care practitioner to monitor and evaluate therapeutic performance. Score database server 404 can also store the results of any questionnaire administered by testing device 402.

When a test subject completes a test (or series of tests) using testing device 402 the results can be provided to score database server 404. The results can include a calculated overall score or provide a detailed response time to each question. Score database server 404 can store the test results in a private database associated with the test subject.

In some embodiments, testing device 402 can further include a method of accepting manual entry of time to complete a test or series of tests. This can allow for non-electronic testing results to be gathered and provided to score database server 404. These manual results can be flagged as a “manual entry” so that this statistical relevance can be used when analyzing the score database.

The private database can be used to compare the test subject's historic results. This can be useful to determine if a test subject's condition is improving or degrading. Applications can include monitoring therapeutic performance to ensure that scores are improving towards an acceptable range. The private database can also include a baseline test result that can be used to evaluate whether the test subject had sustained a concussion. Historic data can also be used to determine progression or diagnosis of any degenerative disease or progression of neurological or neuro-muscular disorders.

In some embodiments, score database server 404 can also anonymize the test results and store these results in a public database. Anonymizing the test results can include removing all personal identifying information but including aspects that can be relevant to the test score, such as age or sex, among other factors. Test results in the public database can be used to compare a test subject's results with norms particular to the test patient. Standard deviation analysis can be performed on the public database to determine these norms for certain classes based on a number of factors, including, but not limited to, age, sex or geographic location. The public database test results can also be used for research and analysis purposes.

Questionnaire data provided by test subjects can also be used to separate public results into normal databases and abnormal databases. For example, test results can be separated based on a pre-existing medical condition, concussive events, medications, learning disabilities, among others.

Score database server 404 can be provided using a high availability web server, such as Nginx, for example, and a database management system, such as Postgre SQL, for example. Communication network 410 can include the internet and score database server 404 can be secured using a firewall. Communication with score database server 404 can be provided over secure socket layer (SSL) protocol, or using other known encryption methods. These can allow score database server 404 to be provided as a secure web service to testing device 402 and access device 406.

Access device 406 can include a computing device 100 with a specific application or a general web browser for connecting with score database server 404. Access device 406 can be used by patients and practitioners to monitor progress and therapeutic conformance. Patients can receive a request or offer to share the private test results with a health care practitioner to allow the practitioner using access device 406 to view results of a patient stored in score data server 404. Score database server 404 can analyze a test subjects private data in order to present information in a manner that is diagnostically useful to the health care practitioner. Typically, this would involve a comparison to normal test results obtained from the public database.

While the exemplary embodiments have been described herein, it is to be understood that the invention is not limited to the disclosed embodiments. The invention is intended to cover various modifications and equivalent arrangements included within the spirit and scope of the appended claims, and scope of the claims is to be accorded an interpretation that encompasses all such modifications and equivalent structures and functions.

1. A method for assessing visual and neuro-cognitive processing, the method comprising:
presenting at least one question on a video display, the question requesting identification of an item position;
receiving at least one response corresponding to the at least one question;
measuring a time period from presenting the at least one question to receiving corresponding response; and
measuring an accuracy of the response to the question based on correct identification of the item position.

2. The method of claim 1, further comprising providing feedback when the response is measured as incorrect.

3. The method of claim 1, wherein a plurality of questions are presented and a plurality of corresponding responses are received.

4. The method of claim 3, further comprising measuring a total time period from presenting the plurality of questions to receiving the plurality of corresponding responses.

5. The method of claim 3, wherein the item position is randomly generated for each of the plurality of questions.

6. The method of claim 1, wherein the response is received by activating a button to measure motor control.

7. The method of claim 1, wherein the response is received by a verbal response received by a microphone.

8. The method of claim 7, wherein the verbal response includes a question number.

9. The method of claim 1, wherein the response is received by a combination of activating a button and receiving a verbal response.

10. The method of claim 1, further comprising comparing the time period to a normative value.

11. The method of claim 10, further comprising providing a questionnaire related to any one of learning disabilities and neurological dysfunction if the time period is outside the normative value.

12. The method of claim 11, wherein the response is received from a test subject and the normative value is based on an age of the test subject.

13. The method of claim 1, further comprising storing the time period in a network accessible database.

14. The method of claim 3, wherein the item position can be any one of left, middle and right, and the response is any one of left, middle and right.

15. The method of claim 3, wherein the item is any one of the words “left”, “right” and “middle”.

16. The method of claim 15, wherein for at least one of the plurality of questions the item position does not correspond to the word.

17. The method of claim 1, wherein the item is an object.

18. The method of claim 17, wherein the object is a geometric shape.

19. The method of claim 1, further comprising calculating a score based on the time period and accuracy.

20. The method of claim 1, further comprising: providing a pair of stereoscopic glasses synchronized with the video display; and wherein the question requesting identification of the item position displaying stereo disparity.

21. A system for assessing visual and neuro-cognitive processing, the system comprising:
a computing device having a video display, a processor and a memory, the memory storing instructions to configure the processor to:
present at least one question on a video display, the question requesting identification of an item position;
receive at least one response corresponding to the at least one question;
measure a time period from presenting the at least one question to receiving corresponding response; and
measure an accuracy of the response to the question based on correct identification of the item position.

22. The system of claim 20, further comprises a network accessible score database, and the computing device further comprises a network interface to send a test score to the score database.

23. The system of claim 20, wherein the network accessible score database has a public “normative” database and an “abnormal” database.

24. The system of claim 20, wherein the network accessible score database has a private database to store a test subject's historic test score.

25. The system of claim 20, wherein the network accessible score database has an interface to allow access to a subject's test scores.
